(19) United States
(12) Patent Application Publication
(10) Pub. No.: US 2026/0216016 A1
(43) Pub. Date: Jul. 30, 2026

(54) MAGNETIC TOOTHPASTE AND MAGNETIC TOOTHBRUSH

(71) Applicants: UNM Rainforest Innovations, Albuquerque, NM (US); The Texas A&M University System, College Station, TX (US); Board of Regents, The University of Texas System, Austin, TX (US)

(72) Inventors: Marek A. Osinski, Albuquerque, NM (US); Hugh D. Smyth, Austin, TX (US); Leisha Marie Martin, Corpus Christi, TX (US); Hennaka M.H.N. Bandara, Bishopsworth, Bristol (GB)

(21) Appl. No.: 19/145,158

(22) PCT Filed: Jan. 2, 2024
(86) PCT No.: PCT/US2024/010040

§ 371 Date: Jul. 1, 2025

Related U.S. Application Data
(60) Provisional application No. 63/436,779, filed on Jan. 3, 2023.

(51) Int. Cl. A61K 8/19; A46B 15/00; A61K 8/21; A61K 8/22; A61K 8/24; A61K 8/25; A61K 8/26; A61Q 11/00

(52) U.S. Cl. CPC A61K 8/19; A46B 15/0026; A61K 8/21; A61K 8/22; A61K 8/24; A61K 8/25; A61K 8/26; A61Q 11/00; A46B 2200/1066; A61K 2800/28; A61K 2800/47; A61K 2800/624

(57) ABSTRACT

A magnetic toothpaste includes magnetic particles having a largest dimension of 1 nm to 2 microns. A magnetic toothbrush includes a handle at one end of the magnetic toothbrush, and a head at an opposite end of the magnetic toothbrush. The head includes a brush. The magnetic toothbrush includes a magnetic field generator.

### CROSS-REFERENCE TO RELATED APPLICATION

This application claims the benefit of priority to U.S. Provisional Patent Application Ser. No. 63/436,779 filed Jan. 3, 2023, the disclosure of which is incorporated herein in its entirety by reference.

### BACKGROUND

Although fluoride alone has been successful in some regard in preventing dental caries, insufficiencies in dental hygiene still exist. Bacterial biofilms can form in the oral cavity, especially on inert surfaces, such as orthodontic wires, bridges, dental fillings, and the like. Bacterial biofilms can cause inflammation and immune dysregulation and lead to gingivitis, periodontitis, bone loss, and tooth loss. Biofilm pores, which are on the order of a few hundred nanometers in size, are resistant to penetration by fluoride despite its small size due to the strong electronegativity of fluoride. In addition, fluoride resistant oral bacteria are common.

### SUMMARY OF THE INVENTION

In various aspects, the present invention provides a magnetic toothpaste that includes magnetic particles having a largest dimension of 1 nm to 2 microns.

In various aspects, the present invention provides a magnetic toothpaste. The magnetic toothpaste includes magnetic particles having a largest dimension of 1 nm to 2 microns. The magnetic particles include ferromagnetic particles, superparamagnetic particles, or a combination thereof. The toothpaste includes an abrasive agent including microparticles that include calcium. The toothpaste also includes a coating on the magnetic particles including alginate and/or alginic acid.

In various aspects, the present invention provides a method of using the toothpaste of the present invention. The method includes contacting a brush of a toothbrush including the magnetic toothpaste on the brush thereof with teeth in an oral cavity.

In various aspects, the present invention provides a tooth-cleaning system that includes the magnetic toothpaste of the present invention. The tooth-cleaning system also includes a toothbrush that includes the magnetic toothpaste on a brush thereof.

In various aspects, the present invention provides a magnetic toothbrush that includes a handle at one end of the magnetic toothbrush. The magnetic toothbrush also includes a head at an opposite end of the magnetic toothbrush. The head includes a brush. The magnetic toothbrush also includes a magnetic field generator.

In various aspects, the present invention provides a kit that includes the magnetic toothbrush of the present invention and that also includes multiple interchangeable heads.

In various aspects, the present invention provides a magnetic toothbrush that includes a handle at one end of the magnetic toothbrush. The magnetic toothbrush includes a head at an opposite end of the magnetic toothbrush. The head includes a brush. The magnetic toothbrush includes a magnetic field generator that generates a rotating magnetic field, a switched magnetic field, or a combination thereof.

In various aspects, the present invention provides a magnetic toothbrush including a handle at one end of the magnetic toothbrush. The magnetic toothbrush also includes a head at an opposite end of the magnetic toothbrush. The head includes a brush. The magnetic toothbrush includes a magnetic field generator that includes a static permanent magnet within the head below bristles of the brush.

In various aspects, the present invention provides a method of using the magnetic toothbrush of the present invention. The method includes contacting the brush of the magnetic toothbrush of the present invention with teeth in an oral cavity.

In various aspects, the present invention provides a method of cleaning teeth. The method includes contacting the brush of the magnetic toothbrush of the present invention that includes the toothpaste of the present invention on the brush thereof with teeth in an oral cavity.

In various aspects, the present invention provides a method of cleaning teeth. The method includes contacting a brush of a magnetic toothbrush including a magnetic toothpaste on the brush thereof with teeth in an oral cavity. The magnetic toothbrush includes a handle at one end of the magnetic toothbrush, and a head at an opposite end of the magnetic toothbrush, the head including the brush. The magnetic toothbrush includes a magnetic field generator that generates a rotating magnetic field, a switched magnetic field, or a combination thereof. The magnetic toothpaste includes magnetic particles having a largest dimension of 1 nm to 2 microns, wherein the magnetic particles include ferromagnetic particles, superparamagnetic particles, or a combination thereof. The magnetic toothpaste includes an abrasive agent including microparticles that include calcium. The magnetic toothpaste also includes a coating on the magnetic particles including alginate and/or alginic acid.

In various aspects, the present invention provides a method of cleaning teeth. The method includes contacting a brush of a magnetic toothbrush including a magnetic toothpaste on the brush thereof with teeth in an oral cavity. The magnetic toothbrush includes a handle at one end of the magnetic toothbrush, and a head at an opposite end of the magnetic toothbrush, the head including a brush. The magnetic toothbrush includes a magnetic field generator that includes a static permanent magnet within the head below bristles of the brush. The magnetic toothpaste includes magnetic particles having a largest dimension of 1 nm to 2 microns, wherein the magnetic particles include ferromagnetic particles, superparamagnetic particles, or a combination thereof. The magnetic toothpaste includes an abrasive agent including microparticles that include calcium. The magnetic toothpaste also includes a coating on the magnetic particles including alginate and/or alginic acid.

In various aspects, the present invention provides a tooth-cleaning system that includes the magnetic toothbrush of the present invention and that includes the magnetic toothpaste of the present invention on the brush thereof.

The present invention relates to the fields of oral hygiene, dentistry, orthodontics and cosmetic dentistry. In various aspects, the magnetic particles in the magnetic toothpaste of the present invention can function as abrasive agents, whitening agent, anti-demineralization agents, biofilm disrupting agents, antimicrobial agents, or a combination thereof. The magnetic particles can remain in an un-agglomerated state during storage of the toothpaste. The magnetic toothpaste and magnetic toothbrush of the present invention, used alone or together, can treat and prevent oral biofilm infections, dental plaque, gingivitis, and/or periodontitis, such as via mechanical biofilm disruption, DNA fragmentation, and/or other mechanisms. The magnetic toothbrush can enhance the magnetic field in the local region and potentially influence the behavior of the magnetic nanoparticles in a synergistic toothpaste formulation.

The magnetic toothbrush and magnetic toothpaste of the present invention can, alone or together, treat oral bacterial, fungal and viral pathogens, including those located in places where typical mechanical devices, brushes, and floss are unable to penetrate, and can provide an enhanced effect in bacterial biofilm eradication.

In various aspects, the magnetic toothpaste of the present invention is not magnetized until used. This avoids agglomeration of the magnetic particles prior to use. By activating the magnetic properties with a toothbrush, the magnetic particles can only become magnetized during use.

A common complication of periodontitis is the onset of periodontal bone loss. In various aspects, electromagnetic fields produced by the magnetic toothbrush of the present invention can also be used to stimulate bone cell proliferation.

The cleaning power of manual and electric toothbrushes is limited by their ability to physically reach to the target area. The magnetic toothbrush of the present invention and or magnetic toothpaste of the present invention can overcomes that limitation by extending the reach of the bristles with a magnetic field which can remotely move the magnetic particles in the magnetic toothpaste to previously unreachable areas of the mouth, such as below the gumline, between the teeth, and/or in and around orthodontic wires, crowns and bridges.

### BRIEF DESCRIPTION OF THE FIGURES

The drawings illustrate generally, by way of example, but not by way of limitation, various aspects of the present invention.

FIG. 1 main image C is a TEM image of magnetic nanoparticles (scale bar is 100 nm), in accordance with various aspects. FIG. 1 inset B is a TEM image of magnetic nanoparticles (scale bar is 10 nm), in accordance with various aspects. FIG. 1 inset A is a fast Fourier transform of a TEM image of magnetic nanoparticles and corresponding miller indices, in accordance with various aspects.

FIG. 2A illustrates a schematic of a magnetic toothbrush, in accordance with various aspects.

FIG. 2B illustrates a diagram of a magnetic toothpaste (e.g., ferropaste) showing magnetically activated “spikes” erupting from the toothbrush, in accordance with various aspects.

FIG. 2C illustrates a magnetic toothpaste (e.g., ferropaste) with a static magnetic field applied thereto, in accordance with various aspects.

FIG. 2D illustrates a magnetic toothpaste with a rotating permanent magnet proximate thereto, in accordance with various aspects.

FIG. 2E illustrates a schematic of a magnetic toothbrush, in accordance with various aspects.

FIG. 3 illustrates a schematic of a magnetic toothpaste in combination a magnetic toothbrush, in accordance with various aspects.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in a specific order as recited herein. Alternatively, in any aspect(s) disclosed herein, specific acts may be carried out in any order without departing from the principles of the invention, except when a temporal or operational sequence is explicitly recited. Furthermore, specified acts can be carried out concurrently unless explicit claim language recites that they be carried out separately or the plain meaning of the claims would require it. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### Magnetic Toothpaste.

In various aspects, the present invention provides a magnetic toothpaste. The magnetic toothpaste includes magnetic particles having a largest dimension of 1 nm to 2 microns. For example, the magnetic particles can have a largest dimension of less than or equal to 2 microns and greater than or equal to 1 nm, 10, 20, 30, 40, 50, 75, 100, 150, 200, 250, 500, 750, 1,000, 1,200, 1,400, 1,600, or 1,800 nm.

The magnetic particles can be any suitable magnetic particles. As used herein, the term “magnetic” means magnetized or magnetizable unless otherwise indicated. The magnetic particles can include magnetic nanoparticles, magnetic microparticles, or a combination thereof. The magnetic particles can include ferrimagnetic particles, ferromagnetic particles, superparamagnetic particles, or a combination thereof. The magnetic particles can include iron, iron oxide, iron nitride, manganese, neodymium, dysprosium, nickel, boron, cobalt, aluminum, or combinations thereof. In the toothpaste, the magnetic particles can be an abrasive agent, a whitening agent, an anti-demineralization agent, a biofilm-disrupting agent, an antimicrobial agent, or a combination thereof. The magnetic particles can form any suitable proportion of the toothpaste, such as 0.01 wt % to 90 wt % of the magnetic toothpaste, or 0.1 wt % to 30 wt %, or 1 wt % to 10 wt %, or 3 wt % to 7 wt %, or less than or equal to 90 wt % and greater than or equal to 0.01 wt %, 0.05, 0.1, 0.5, 1, 2, 3, 4, 5, 6, 7, 8, 9, 10, 12, 14, 16, 18, 20, 25, 30, 35, 40, 45, 50, 55, 60, 65, 70, 75, 80, or 85 wt %. The magnetic particles can include a polymer coating. The polymer coating can be any suitable polymer coating. The polymer coating can include a hydrocarbon, a silicate, or a combination thereof. The polymer coating can include dextran, alginate, or a combination thereof. The coating can completely coat the magnetic particles.

In various aspects, the magnetic toothpaste can be free of abrasive agents other than the magnetic particles. In various aspects, the magnetic toothpaste can include one or more abrasive agents. The abrasive agent can assist in mechanical cleaning of surfaces of the teeth. The abrasive agent can be any suitable abrasive agent, such as an abrasive agent including calcium, ground natural chalk, calcium carbonate, dicalcium phosphate dihydrate, silica, alumina, or a combination thereof. The abrasive agent can include microparticles, nanoparticles, or a combination thereof. The abrasive agent can form any suitable proportion of the magnetic toothpaste, such as 1 wt % to 90 wt % of the magnetic toothpaste, 10 wt % to 75 wt %, or less than or equal to 90 wt % and greater than or equal to 1 wt %, 2, 4, 6, 8, 10, 12, 14, 16, 18, 20, 25, 30, 35, 40, 45, 50, 55, 60, 65, 70, 75, 80, or 85 wt % of the magnetic toothpaste.

In various aspects, the magnetic toothpaste can be free of fluoride salts. In various aspects, the magnetic toothpaste can include on or more fluoride salts, such as a fluoride salt including sodium monofluorophosphate (SMFP), sodium fluoride, stannous fluoride, or a combination thereof.

The magnetic toothpaste can include a carrier fluid. The carrier fluid can include any suitable one or more fluids, such as water, polyethylene glycol, glycerin, propylene glycol, or a combination thereof. The carrier fluid can form any suitable proportion of the magnetic toothpaste, such as 1 wt % to 90 wt % of the magnetic toothpaste, 5 wt % to 75 wt %, or less than or equal to 90 wt % and greater than or equal to 1 wt %, 2, 4, 6, 8, 10, 12, 14, 16, 18, 20, 25, 30, 35, 40, 45, 50, 55, 60, 65, 70, 75, 80, or 85 wt % of the magnetic toothpaste. Water can form any suitable proportion of the magnetic toothpaste, such as 5 wt % to 75 wt %.

The magnetic toothpaste can further include hydrogen peroxide, carbamide peroxide, or a combination thereof. The hydrogen peroxide and/or carbamide peroxide can form any suitable proportion of the magnetic toothpaste 0.1 wt % to 10 wt % of the magnetic toothpaste, or 0.2 wt % to 5 wt %.

The magnetic toothpaste can include a flavoring agent. The flavoring agent can include any suitable one or more flavors, and can be form any suitable proportion of the magnetic toothpaste, such as 0.01 wt % to 30 wt %, or 0.1 wt % to 2.5 wt %.

The magnetic toothpaste can include one or more humectants. The humectant can be any suitable humectant, such as sorbitol, xylitol, polyethylene glycol, glycerin, propylene glycol, or a combination thereof. The one or more humectants can form any suitable proportion of the magnetic toothpaste, such as 1 wt % to 70 wt % of the toothpaste, or 2 wt % to 50 wt % of the toothpaste.

The magnetic toothpaste can further include one or more sweeteners, binders, antimicrobial agents, preservatives, thickeners, binders, glossing agents, colorants, antifoam agents, foaming agents, surfactants, emulsifiers, or a combination thereof.

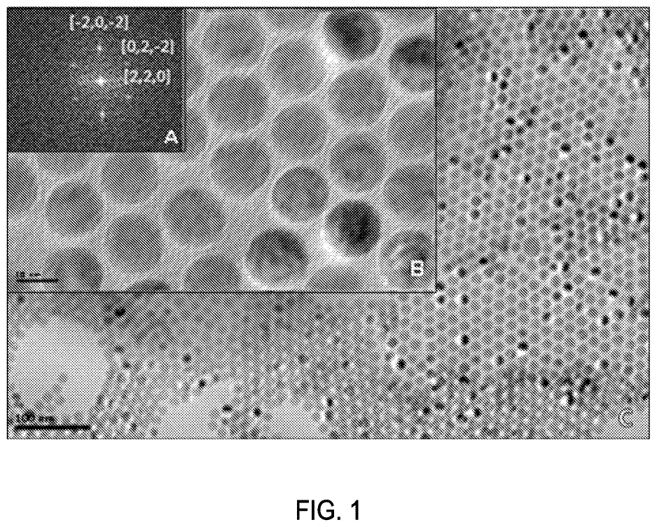

FIG. 1 main image C is a TEM image of magnetic nanoparticles (scale bar is 100 nm). FIG. 1 inset B is a TEM image of magnetic nanoparticles (scale bar is 10 nm). FIG. 1 inset A is a fast Fourier transform of a TEM image of magnetic nanoparticles and corresponding miller indices. The nanoparticles shown in FIG. 1 main image C and insets B and A are multi-phase iron oxide, including spinel phase (Fe₃O₄/Fe₂O₃) and Wustite phase (FeO).

### Method of Using the Magnetic Toothpaste.

Various aspects of the present invention provide a method of using the magnetic toothpaste of the present invention. The method can include contacting a brush of a toothbrush including the magnetic toothpaste on the brush thereof with teeth in an oral cavity. The toothbrush can be any suitable type of toothbrush, such as the magnetic toothbrush of the present invention, another magnetic toothbrush, or a non-magnetic toothbrush.

### Tooth-Cleaning System.

Various aspects of the present invention provide a tooth-cleaning system. The tooth-cleaning system can include the magnetic toothpaste of the present invention. The system can also include a toothbrush, wherein the toothbrush includes a the magnetic toothpaste on a brush thereof. The toothbrush can be any suitable type of toothbrush, such as the magnetic toothbrush of the present invention, another magnetic toothbrush, or a non-magnetic toothbrush.

### Magnetic Toothbrush.

In various aspects the present invention provide a magnetic toothbrush. The magnetic toothbrush includes a handle at one end of the magnetic toothbrush. The magnetic toothbrush includes a head at an opposite end of the magnetic toothbrush (i.e., an end opposite from the end that includes the handle). The magnetic toothbrush also include a magnetic field generator.

The magnetic field generator can include a permanent magnet, an electromagnet, or a combination thereof. The magnetic field generator can generate a magnetic field (i.e., when measured with the magnetic toothbrush in a stationary state) that includes a static magnetic field, a switched magnetic field, an oscillating magnetic field, a rotating magnetic field, or a combination thereof. The magnetic field generator can generate a magnetic field that includes a rotating magnetic field, such as with a rotator (e.g., a motor) that rotates a permanent magnet or electromagnet.

The magnetic field generator can include a permanent magnet. The permanent magnet can include (as measured with the magnetic toothbrush in a static position) a static permanent magnet, a rotating permanent magnet, an oscillating permanent magnet, a vibrating permanent magnet, or a combination thereof. The permanent magnet can be a static magnet. The permanent magnet can be a rotating magnet; for example, the toothbrush can include a rotator that rotates that permanent magnet. The toothbrush can include a vibrator or oscillator that vibrates and/or oscillates the permanent magnet. The permanent magnet can include any suitable permanent magnet, such as an AlNiCo magnet, a SmCo magnet, a ceramic magnet, a NdFeB magnet, or a combination thereof. The permanent magnet can include Nd.

The magnetic field generator can include an electromagnet. The electromagnet can be (as measured with the magnetic toothbrush in a stationary position) a static electromagnet, a rotating electromagnet, an oscillating electromagnet, a vibrating electromagnet, or a combination thereof. The electromagnet can be a static electromagnet. The electromagnet can be a rotating electromagnet. The toothbrush can include a rotator (e.g., a motor) that rotates the electromagnet. The toothbrush can include a vibrator or oscillator that vibrates and/or oscillates the electromagnet.

The magnetic field generator can include a single permanent magnet or a single electromagnet. The magnetic field generator can include multiple permanent magnets or multiple electromagnets.

The magnetic field generator can be located in any suitable one or more portions of the magnetic toothbrush. The magnetic field generator can be located within the head of the magnetic toothbrush. The head can include the magnetic field generator at a portion of the head that includes the brush, such as below the bristles of the brush. In some aspects, the handle of the toothbrush can include the magnetic field generator. In various aspects, both the handle and the head of the magnetic tooth brush include the magnetic field generator.

In various aspects, the magnetic field generator can include a static permanent magnet within the head below bristles of the brush. In some examples, the permanent magnet can be oriented with the north end or the south end of the permanent magnet facing toward the bristles of the brush.

In various aspects, the magnetic field generator can include a rotating permanent magnet within the head below bristles of the brush. The magnetic toothbrush can include a rotator that rotates the permanent magnet.

In various aspects, the magnetic toothbrush can include static bristles and a static brush. The magnetic field generator includes a rotating, vibrating, and/or oscillating permanent magnet or electromagnet. The magnetic field generator can include a static magnet or electromagnet.

In various aspects, the magnetic toothbrush can include vibrating and/or oscillating bristles and/or brush. The magnetic field generator can include a rotating, vibrating, and/or oscillating permanent magnet or electromagnet. The magnetic field generator can include a static magnet or electromagnet.

In various aspects, the magnetic toothbrush can be an electric toothbrush. The magnetic toothbrush can include a battery.

In various aspects, the magnetic toothbrush can be free of batteries, and the magnetic toothbrush can be a manual toothbrush.

In various aspect, the magnetic toothbrush can include multiple interchangeable heads. The multiple interchangeable heads can be the same or can include different types of magnetic field generators. In various aspects, the present invention provides a kit including the magnetic toothbrush of the present invention and multiple interchangeable heads (e.g., at least one interchangeable head in addition to the head of the magnetic toothbrush).

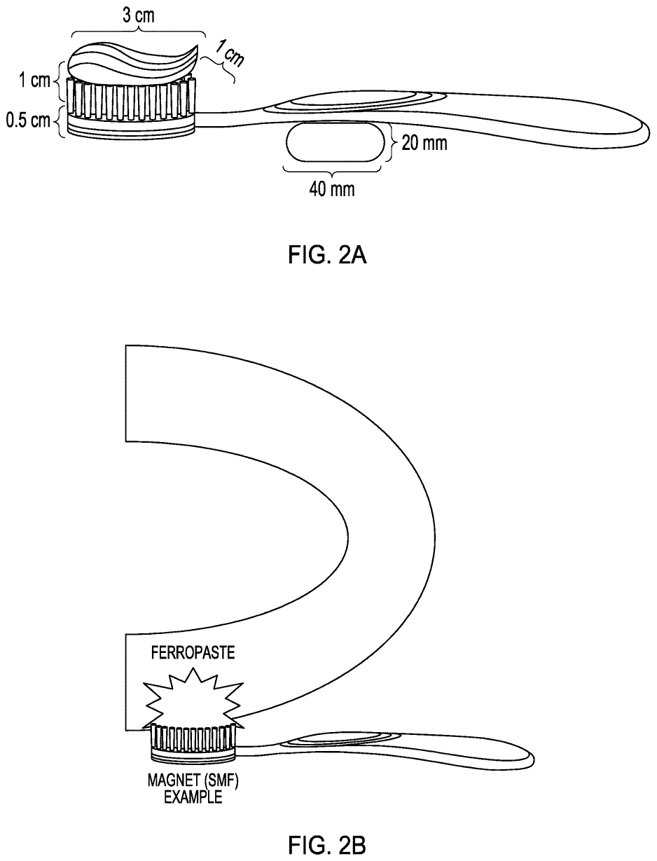

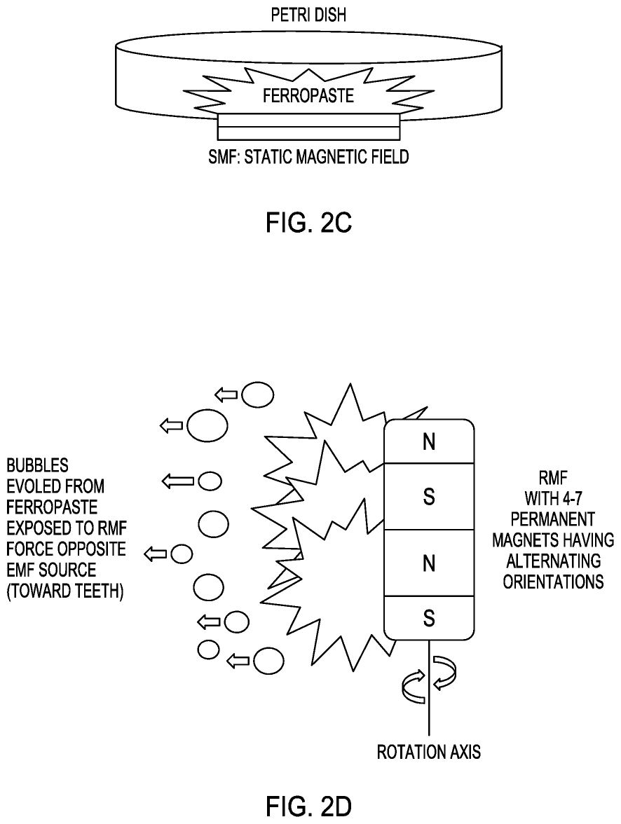

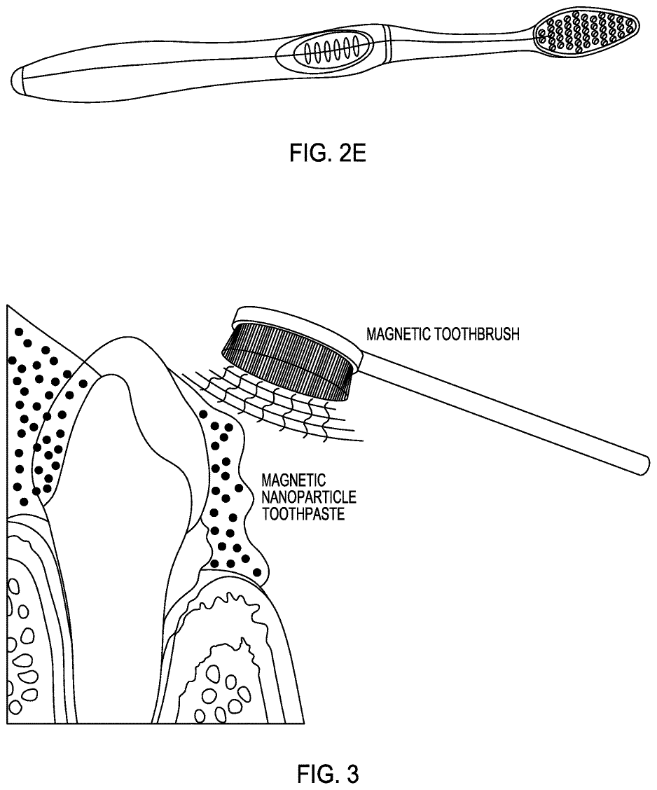

FIG. 2A illustrates a schematic of a magnetic toothbrush that illustrates various dimensions thereof and includes a magnetic toothpaste (e.g., ferropaste) thereon. FIG. 2B illustrates a diagram of a magnetic toothpaste (e.g., ferropaste) on a toothbrush that is brushing teeth and that is exposed to a static magnetic field, showing magnetically activated “spikes” erupting from the toothpaste. FIG. 2C illustrates a magnetic toothpaste (e.g., ferropaste) in a petri dish being exposed to a static magnetic field, showing magnetically activated “spikes” erupting from the toothpaste. FIG. 2D illustrates a magnetic toothpaste with a rotating permanent magnet proximate thereto, illustrating bubbles evolved from the ferropaste that is exposed to the rotating magnetic field which provides a force opposite the magnetic field source (e.g., a force toward the teeth). FIG. 2E illustrates a schematic of a magnetic toothbrush that includes a static permanent magnet in the head thereof.

FIGS. 2B and 2E illustrate a manual magnetic toothbrush including a handle and a static (permanent magnet) in the head. The permanent magnet can include any suitable ferro- or ferri-magnetic material and may have any suitable field orientation. In one aspect, the front face (beneath the bristles) is the north end of the magnet. The static magnet can manipulate the magnetic toothpaste as the user manually manipulates the brush to clean their teeth.

In various aspects, the magnetic toothbrush can include a static magnetic field of ~13,200 Gauss, 42 MGOe. In various aspects, a switched magnetic field can be produced by a user using the magnetic toothbrush on an outer surface of teeth followed by an inner surface or vice versa. In various aspects, the toothbrush head includes four ~ 1/16″ NdFeB (permanent) magnets, that can have a surface field of ~5754 Gauss, attached to a rotor. Two magnets can be oriented axially, mounted in opposite orientations and two are oriented diametrically, mounted in opposite orientations. The orientations can be such that the field is inconsistent and results in crossing field lines. The rotation of the magnets in the head can further change the field. Any suitable rotation speed can be used; for example, 1,000 RPM can be used to activate the nanoparticles without allowing time for the nanoparticles to migrate towards the head.

### Method of Using the Magnetic Toothbrush.

Various aspects of the present invention provide method of using the magnetic toothbrush of the present invention. The method includes contacting the brush of the magnetic toothbrush of the present invention with teeth in an oral cavity. The brush can include toothpaste. The toothpaste can be any suitable toothpaste, such as a non-magnetic toothpaste, or the magnetic toothpaste of the present invention, or a magnetic toothpaste other than that of the present invention.

### Method of Cleaning Teeth.

Various aspects of the present invention provide a method of cleaning teeth. The method includes contacting the brush of the magnetic toothbrush of the present invention including the magnetic toothpaste of the present invention on the brush thereof with teeth in an oral cavity.

### Tooth-Cleaning System.

Various aspects of the present invention provide a tooth-cleaning system. The tooth-cleaning system can include the magnetic toothpaste of the present invention. The tooth-cleaning system can also include the magnetic toothbrush of the present invention including the magnetic toothpaste on the brush thereof.

FIG. 3 illustrates a schematic of a magnetic toothpaste in combination with a magnetic toothbrush, in accordance with various aspects. The magnetic toothbrush includes a static permanent magnet in the head thereof, and the toothbrush is shown brushing a tooth that protrudes from gums.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects.

The following exemplary aspects are provided, the numbering of which is not to be construed as designating levels of importance:

Aspect 1 provides a magnetic toothpaste comprising:
magnetic particles having a largest dimension of 1 nm to 2 microns.

Aspect 2 provides the magnetic toothpaste of Aspect 1, wherein the magnetic particles comprise nanoparticles.

Aspect 3 provides the magnetic toothpaste of any one of Aspects 1-2, wherein the magnetic particles comprise microparticles.

Aspect 4 provides the magnetic toothpaste of any one of Aspects 1-3, wherein the magnetic particles comprise ferrimagnetic particles, ferromagnetic particles, superparamagnetic particles, or a combination thereof.

Aspect 5 provides the magnetic toothpaste of any one of Aspects 1-4, wherein the magnetic particles comprise iron, iron oxide, iron nitride, manganese, neodymium, dysprosium, nickel, boron, cobalt, aluminum, or combinations thereof.

Aspect 6 provides the magnetic toothpaste of any one of Aspects 1-5, wherein the magnetic particles are an abrasive agent, a whitening agent, an anti-demineralization agent, a biofilm-disrupting agent, an antimicrobial agent, or a combination thereof.

Aspect 7 provides the magnetic toothpaste of any one of Aspects 1-6, wherein the magnetic particles are 0.01 wt % to 90 wt % of the magnetic toothpaste.

Aspect 8 provides the magnetic toothpaste of any one of Aspects 1-7, wherein the magnetic particles are 1 wt % to 10 wt % of the magnetic toothpaste.

Aspect 9 provides the magnetic toothpaste of any one of Aspects 1-8, wherein the magnetic particles comprise a polymer coating.

Aspect 10 provides the magnetic toothpaste of Aspect 9, wherein the polymer coating comprises a hydrocarbon, a silicate, or a combination thereof.

Aspect 11 provides the magnetic toothpaste of any one of Aspects 9-10, wherein the polymer coating comprises dextran, alginate, or a combination thereof.

Aspect 12 provides the magnetic toothpaste of any one of Aspects 1-11, wherein the toothpaste is free of abrasive agents other than the magnetic particles.

Aspect 13 provides the magnetic toothpaste of any one of Aspects 1-11, further comprising an abrasive agent.

Aspect 14 provides the magnetic toothpaste of Aspect 13, wherein the abrasive agent comprises calcium, ground natural chalk, calcium carbonate, dicalcium phosphate dihydrate, silica, alumina, or a combination thereof.

Aspect 15 provides the magnetic toothpaste of any one of Aspects 13-14, wherein the abrasive agent comprises microparticles, nanoparticles, or a combination thereof.

Aspect 16 provides the magnetic toothpaste of any one of Aspects 13-15, wherein the abrasive agent is 1 wt % to 90 wt % of the magnetic toothpaste.

Aspect 17 provides the magnetic toothpaste of any one of Aspects 13-16, wherein the abrasive agent is 10 wt % to 75 wt % of the magnetic toothpaste.

Aspect 18 provides the magnetic toothpaste of any one of Aspects 1-17, further comprising a fluoride salt.

Aspect 19 provides the magnetic toothpaste of Aspect 18, wherein the fluoride salt comprises sodium monofluorophosphate (SMFP), sodium fluoride, stannous fluoride, or a combination thereof.

Aspect 20 provides the magnetic toothpaste of any one of Aspects 1-19, further comprising a carrier fluid.

Aspect 21 provides the magnetic toothpaste of Aspect 20, wherein the carrier fluid comprises water, polyethylene glycol, glycerin, propylene glycol, or a combination thereof.

Aspect 22 provides the magnetic toothpaste of any one of Aspects 20-21, wherein the carrier fluid is 1 wt % to 90 wt % of the magnetic toothpaste.

Aspect 23 provides the magnetic toothpaste of any one of Aspects 20-22, wherein the carrier fluid is 5 wt % to 75 wt % of the magnetic toothpaste.

Aspect 24 provides the magnetic toothpaste of any one of Aspects 1-23, wherein water is 5 wt % to 75 wt % of the magnetic toothpaste.

Aspect 25 provides the magnetic toothpaste of any one of Aspects 1-24, further comprising hydrogen peroxide, carbamide peroxide, or a combination thereof.

Aspect 26 provides the magnetic toothpaste of Aspect 25, wherein the hydrogen peroxide and/or carbamide peroxide is 0.1 wt % to 10 wt % of the magnetic toothpaste.

Aspect 27 provides the magnetic toothpaste of any one of Aspects 25-26, wherein the hydrogen peroxide and/or carbamide peroxide is 0.2 wt % to 5 wt % of the magnetic toothpaste.

Aspect 28 provides the magnetic toothpaste of any one of Aspects 1-27, further comprising a flavoring agent.

Aspect 29 provides the magnetic toothpaste of Aspect 28, wherein the flavoring agent is 0.01 wt % to 30 wt % of the magnetic toothpaste.

Aspect 30 provides the magnetic toothpaste of any one of Aspects 28-29, wherein the flavoring agent is 0.1 wt % to 2.5 wt % of the magnetic toothpaste.

Aspect 31 provides the magnetic toothpaste of any one of Aspects 1-30, further comprising one or more humectants.

Aspect 32 provides the toothpaste of Aspect 31, wherein the humectant comprises sorbitol, xylitol, polyethylene glycol, glycerin, propylene glycol, or a combination thereof.

Aspect 33 provides the toothpaste of any one of Aspects 31-32, wherein the one or more humectants are 1 wt % to 70 wt % of the toothpaste.

Aspect 34 provides the toothpaste of any one of Aspects 31-33, wherein the one or more humectants are 2 wt % to 50 wt % of the toothpaste.

Aspect 35 provides the toothpaste of any one of Aspects 1-34, further comprising one or more sweeteners, binders, antimicrobial agents, preservatives, thickeners, binders, glossing agents, colorants, antifoam agents, foaming agents, surfactants, emulsifiers, or a combination thereof.

Aspect 36 provides a magnetic toothpaste comprising:
magnetic particles having a largest dimension of 1 nm to 2 microns, wherein the magnetic particles comprise ferromagnetic particles, superparamagnetic particles, or a combination thereof;
an abrasive agent comprising microparticles that comprise calcium; and
a coating on the magnetic particles comprising alginate and/or alginic acid.

Aspect 37 provides a method of using the magnetic toothpaste of any one of Aspects 1-36, the method comprising:
contacting a brush of a toothbrush comprising the magnetic toothpaste on the brush thereof with teeth in an oral cavity.

Aspect 38 provides a tooth-cleaning system comprising:
the magnetic toothpaste of any one of Aspects 1-36; and
a toothbrush, wherein the toothbrush comprises the toothpaste on a brush thereof.

Aspect 39 provides a magnetic toothbrush comprising:
a handle at one end of the magnetic toothbrush; and
a head at an opposite end of the magnetic toothbrush, the head comprising a brush;
wherein the magnetic toothbrush comprises a magnetic field generator.

Aspect 40 provides the magnetic toothbrush of Aspect 39, wherein the magnetic field generator comprises a permanent magnet, an electromagnet, or a combination thereof.

Aspect 41 provides the magnetic toothbrush of any one of Aspects 39-40, wherein the magnetic field generator generates a magnetic field that comprises a static magnetic field, a switched magnetic field, an oscillating magnetic field, a rotating magnetic field, or a combination thereof.

Aspect 42 provides the magnetic toothbrush of any one of Aspects 39-41, wherein the magnetic field generator generates a magnetic field that comprises a rotating magnetic field.

Aspect 43 provides the magnetic toothbrush of any one of Aspects 39-42, wherein the magnetic field generator comprises a permanent magnet.

Aspect 44 provides the magnetic toothbrush of Aspect 43, wherein the permanent magnet is a static permanent magnet, a rotating permanent magnet, an oscillating permanent magnet, a vibrating permanent magnet, or a combination thereof.

Aspect 45 provides the magnetic toothbrush of any one of Aspects 43-44, wherein the permanent magnet is a static magnet.

Aspect 46 provides the magnetic toothbrush of any one of Aspects 43-45, wherein the permanent magnet is a rotating magnet.

Aspect 47 provides the magnetic toothbrush of any one of Aspects 43-46, wherein the toothbrush comprises a rotator that rotates that permanent magnet.

Aspect 48 provides the magnetic toothbrush of any one of Aspects 43-47, wherein toothbrush comprises a vibrator or oscillator that vibrates and/or oscillates the permanent magnet.

Aspect 49 provides the magnetic toothbrush of any one of Aspects 43-48, wherein the permanent magnet comprises an AlNiCo magnet, a SmCo magnet, a ceramic magnet, a NdFeB magnet, or a combination thereof.

Aspect 50 provides the magnetic toothbrush of any one of Aspects 43-49, wherein the permanent magnet comprises Nd.

Aspect 51 provides the magnetic toothbrush of any one of Aspects 39-50, wherein the magnetic field generator comprises an electromagnet.

Aspect 52 provides the magnetic toothbrush of Aspect 51, wherein the electromagnet comprises a static electromagnet, a rotating electromagnet, an oscillating electromagnet, a vibrating electromagnet, or a combination thereof.

Aspect 53 provides the magnetic toothbrush of any one of Aspects 51-52, wherein the electromagnet is a static magnet.

Aspect 54 provides the magnetic toothbrush of any one of Aspects 51-53, wherein the electromagnet is a rotating magnet.

Aspect 55 provides the magnetic toothbrush of any one of Aspects 51-54, wherein the toothbrush comprises a rotator that rotates that electromagnet.

Aspect 56 provides the magnetic toothbrush of any one of Aspects 51-55, wherein the toothbrush comprises a vibrator or oscillator that vibrates and/or oscillates the electromagnet.

Aspect 57 provides the magnetic toothbrush of any one of Aspects 39-56, wherein the magnetic field generator comprises a single magnet or electromagnet.

Aspect 58 provides the magnetic toothbrush of any one of Aspects 39-57, wherein the magnetic field generator comprises more than one magnet or electromagnet.

Aspect 59 provides the magnetic toothbrush of any one of Aspects 39-58, wherein the head comprises the magnetic field generator.

Aspect 60 provides the magnetic toothbrush of Aspect 59, wherein the head comprises the magnetic field generator at a portion of the head that comprises the brush.

Aspect 61 provides the magnetic toothbrush of any one of Aspects 59-60, wherein the head comprises the magnetic field generator within the head below bristles of the brush.

Aspect 62 provides the magnetic toothbrush of any one of Aspects 39-61, wherein the handle comprises the magnetic field generator.

Aspect 63 provides the magnetic toothbrush of any one of Aspects 39-62, wherein both the head and the handle comprise the magnetic field generator.

Aspect 64 provides the magnetic toothbrush of any one of Aspects 39-63, wherein the magnetic field generator comprises a static permanent magnet within the head below bristles of the brush.

Aspect 65 provides the magnetic toothbrush of Aspect 64, wherein the permanent magnet is oriented with a north end of the permanent magnet facing the bristles.

Aspect 66 provides the magnetic toothbrush of any one of Aspects 39-63, wherein the magnetic field generator comprises a rotating permanent magnet within the head below bristles of the brush, wherein the toothbrush comprises a rotator that rotates the permanent magnet.

Aspect 67 provides the magnetic toothbrush of any one of Aspects 39-66, comprising static bristles and a static brush.

Aspect 68 provides the magnetic toothbrush of Aspect 67, wherein the magnetic field generator comprises a rotating, vibrating, and/or oscillating permanent magnet or electromagnet.

Aspect 69 provides the magnetic toothbrush of any one of Aspects 67-68, wherein the magnetic field generator comprises a static magnet or electromagnet.

Aspect 70 provides the magnetic toothbrush of any one of Aspects 39-69, wherein the magnetic toothbrush comprising vibrating and/or oscillating bristles and/or brush.

Aspect 71 provides the magnetic toothbrush of Aspect 70, wherein the magnetic field generator comprises a rotating, vibrating, and/or oscillating permanent magnet or electromagnet.

Aspect 72 provides the magnetic toothbrush of any one of Aspects 70-71, wherein the magnetic field generator comprises a static magnet or electromagnet.

Aspect 73 provides the magnetic toothbrush of any one of Aspects 39-72, wherein the magnetic toothbrush is an electric toothbrush.

Aspect 74 provides the magnetic toothbrush of Aspect 73, wherein the magnetic toothbrush comprises a battery.

Aspect 75 provides the magnetic toothbrush of any one of Aspects 39-74, wherein the magnetic toothbrush is free of batteries, wherein the magnetic toothbrush is a manual toothbrush.

Aspect 76 provides the magnetic toothbrush of any one of Aspects 39-75, wherein the magnetic toothbrush comprises multiple interchangeable heads.

Aspect 77 provides the magnetic toothbrush of any one of Aspects 39-76, wherein the multiple interchangeable heads comprise different magnetic field generators.

Aspect 78 provides a kit comprising the magnetic toothbrush of any one of Aspects 39-77 and multiple interchangeable heads.

Aspect 79 provides a magnetic toothbrush comprising:
a handle at one end of the magnetic toothbrush; and
a head at an opposite end of the magnetic toothbrush, the head comprising a brush;
wherein the magnetic toothbrush comprises a magnetic field generator that generates a rotating magnetic field, a switched magnetic field, or a combination thereof.

Aspect 80 provides a magnetic toothbrush comprising:
a handle at one end of the magnetic toothbrush; and
a head at an opposite end of the magnetic toothbrush, the head comprising a brush;
wherein the magnetic toothbrush comprises a magnetic field generator that comprises a static permanent magnet within the head below bristles of the brush.

Aspect 81 provides a method of using the magnetic toothbrush of any one of Aspects 39-77, the method comprising:
contacting the brush of the magnetic toothbrush of any one of Aspects 39-77 with teeth in an oral cavity.

Aspect 82 provides the method of Aspect 81, wherein the brush comprises toothpaste.

Aspect 83 provides the method of Aspect 82, wherein the toothpaste comprises a magnetic toothpaste.

Aspect 84 provides the method of Aspect 82, wherein the toothpaste comprises a non-magnetic toothpaste.

Aspect 85 provides a method of cleaning teeth, the method comprising:
contacting the brush of the magnetic toothbrush of any one of Aspects 39-77 comprising the magnetic toothpaste of any one of Aspects 1-36 on the brush thereof with teeth in an oral cavity.

Aspect 86 provides a method of cleaning teeth, the method comprising:
contacting a brush of a magnetic toothbrush comprising a magnetic toothpaste on the brush thereof with teeth in an oral cavity;
wherein the magnetic toothbrush comprises
   a handle at one end of the magnetic toothbrush, and
   a head at an opposite end of the magnetic toothbrush, the head comprising the brush,
   wherein the magnetic toothbrush comprises a magnetic field generator that generates a rotating magnetic field, a switched magnetic field, or a combination thereof, and
wherein the magnetic toothpaste comprises
   magnetic particles having a largest dimension of 1 nm to 2 microns, wherein the magnetic particles comprise ferromagnetic particles, superparamagnetic particles, or a combination thereof,
   an abrasive agent comprising microparticles that comprise calcium, and
   a coating on the magnetic particles comprising alginate and/or alginic acid.

Aspect 87 provides a method of cleaning teeth, the method comprising:
contacting a brush of a magnetic toothbrush comprising a magnetic toothpaste on the brush thereof with teeth in an oral cavity;
wherein the magnetic toothbrush comprises
   a handle at one end of the magnetic toothbrush, and
   a head at an opposite end of the magnetic toothbrush, the head comprising a brush,
   wherein the magnetic toothbrush comprises a magnetic field generator that comprises a static permanent magnet within the head below bristles of the brush; and
wherein the magnetic toothpaste comprises
   magnetic particles having a largest dimension of 1 nm to 2 microns, wherein the magnetic particles comprise ferromagnetic particles, superparamagnetic particles, or a combination thereof,
   an abrasive agent comprising microparticles that comprise calcium, and
   a coating on the magnetic particles comprising alginate and/or alginic acid.

Aspect 88 provides a tooth-cleaning system comprising:
the magnetic toothpaste of any one of Aspects 1-36; and
the magnetic toothbrush of any one of Aspects 39-77 comprising the magnetic toothpaste on the brush thereof.

Aspect 89 provides the magnetic toothpaste, magnetic toothbrush, method, or system of any one or any combination of Aspects 1-88 optionally configured such that all elements or options recited are available to use or select from.

1. A magnetic toothpaste comprising:
magnetic particles having a largest dimension of 1 nm to 2 microns.

2. The magnetic toothpaste of claim 1, wherein the magnetic particles comprise ferrimagnetic particles, ferromagnetic particles, superparamagnetic particles, or a combination thereof.

3. The magnetic toothpaste of claim 1, wherein the magnetic particles are 1 wt % to 10 wt % of the magnetic toothpaste.

4. The magnetic toothpaste of claim 1, wherein the magnetic particles comprise a polymer coating.

5. The magnetic toothpaste of claim 1, further comprising an abrasive agent, wherein the abrasive agent comprises calcium, ground natural chalk, calcium carbonate, dicalcium phosphate dihydrate, silica, alumina, or a combination thereof.

6. The magnetic toothpaste of claim 1, further comprising a fluoride salt, a carrier fluid, hydrogen peroxide, carbamide peroxide, and/or one or more flavoring agents, humectants, one or more sweeteners, binders, antimicrobial agents, preservatives, thickeners, binders, glossing agents, colorants, antifoam agents, foaming agents, surfactants, emulsifiers, or a combination thereof.

7. The magnetic toothpaste of claim 1, wherein the magnetic particles comprise ferromagnetic particles, superparamagnetic particles, or a combination thereof, and wherein the magnetic toothpaste further comprises:
an abrasive agent comprising microparticles that comprise calcium; and
a coating on the magnetic particles comprising alginate and/or alginic acid.

8. A method of using the magnetic toothpaste of claim 1, the method comprising:
contacting a brush of a toothbrush comprising the magnetic toothpaste on the brush thereof with teeth in an oral cavity.

9. A tooth-cleaning system comprising:
the magnetic toothpaste of claim 1; and
a toothbrush, wherein the toothbrush comprises the magnetic toothpaste on a brush thereof.

10. A magnetic toothbrush comprising:
a handle at one end of the magnetic toothbrush; and
a head at an opposite end of the magnetic toothbrush, the head comprising a brush;
wherein the magnetic toothbrush comprises a magnetic field generator.

11. The magnetic toothbrush of claim 10, wherein the magnetic field generator comprises a permanent magnet, an electromagnet, or a combination thereof.

12. The magnetic toothbrush of claim 10, wherein the magnetic field generator generates a magnetic field that comprises a static magnetic field, a switched magnetic field, an oscillating magnetic field, a rotating magnetic field, or a combination thereof.

13. The magnetic toothbrush of claim 10, wherein the magnetic field generator generates a magnetic field that comprises a rotating magnetic field.

14. The magnetic toothbrush of claim 10, wherein the magnetic field generator generates a rotating magnetic field, a switched magnetic field, or a combination thereof.

15. The magnetic toothbrush of claim 10, wherein the magnetic field generator comprises a static permanent magnet within the head below bristles of the brush.

16. A method of using the magnetic toothbrush of claim 10, the method comprising:
contacting the brush of the magnetic toothbrush with teeth in an oral cavity.

17. A method of cleaning teeth, the method comprising:
contacting the brush of the magnetic toothbrush of claim 10 comprising magnetic toothpaste on the brush thereof with teeth in an oral cavity, the magnetic toothpaste comprising magnetic particles having a largest dimension of 1 nm to 2 microns.

18. A method of cleaning teeth, the method comprising:
contacting the brush of the magnetic toothbrush of claim 14 comprising a magnetic toothpaste on the brush thereof with teeth in an oral cavity;
wherein the magnetic toothpaste comprises
magnetic particles having a largest dimension of 1 nm to 2 microns, wherein the magnetic particles comprise ferromagnetic particles, superparamagnetic particles, or a combination thereof,
an abrasive agent comprising microparticles that comprise calcium, and
a coating on the magnetic particles comprising alginate and/or alginic acid.

19. A method of cleaning teeth, the method comprising:
contacting a brush of a magnetic toothbrush comprising a magnetic toothpaste on the brush thereof with teeth in an oral cavity;
wherein the magnetic toothbrush comprises
a handle at one end of the magnetic toothbrush, and
a head at an opposite end of the magnetic toothbrush, the head comprising a brush,
wherein the magnetic toothbrush comprises a magnetic field generator that comprises a static permanent magnet within the head below bristles of the brush; and
wherein the magnetic toothpaste comprises
magnetic particles having a largest dimension of 1 nm to 2 microns, wherein the magnetic particles comprise ferromagnetic particles, superparamagnetic particles, or a combination thereof,
an abrasive agent comprising microparticles that comprise calcium, and
a coating on the magnetic particles comprising alginate and/or alginic acid.

20. A tooth-cleaning system comprising:
a magnetic toothpaste comprising magnetic particles having a largest dimension of 1 nm to 2 microns; and
the magnetic toothbrush of claim 10 comprising the magnetic toothpaste on the brush thereof.
